(19) United States
(12) Patent Application Publication
(10) Pub. No.: US 2024/0123086 A1
(43) Pub. Date: Apr. 18, 2024

(54) INDUCIBLE SINGLE AAV SYSTEM AND USES THEREOF

(71) Applicant: University of Massachusetts One Beacon Street, Boston, MA (US)

(72) Inventor: Christian Mueller, Worcester, MA (US)

(73) Assignee: University of Massachusetts, Boston, MA (US)

(21) Appl. No.: 18/278,260

(22) PCT Filed: Feb. 24, 2022
(86) PCT No.: PCT/US2022/017689

§ 371 Date: Aug. 22, 2023

(51) Int. Cl. A61K 48/00; A61K 31/7036; C12N 9/02; C12N 9/90; C12N 15/86

(52) U.S. Cl. CPC A61K 48/005; A61K 31/7036; C12N 9/0071; C12N 9/90; C12N 15/86; C12N 2740/15043; C12Y 114/11027; C12Y 502/01008

(57) ABSTRACT

Aspects of the disclosure relate to compositions and methods for epigenetic regulation of endogenous gene expression from viral vectors. In some embodiments, the disclosure provides expression constructs comprising a viral vector encoding a transgene, the activation of which is regulated by a rapamycin/rapalog-based system, and the transgene is capable of epigenetically regulate an endogenous gene.

### RELATED APPLICATIONS

This application is a national stage filing under 35 U.S.C. § 371 of international PCT application PCT/US2022/017689, filed Feb. 24, 2022, which claims priority under 35 U.S.C. § 119(e) to U.S. provisional patent application, U.S. Ser. No. 63/153,268, filed Feb. 24, 2021, the entire contents of each of which are incorporated by reference herein.

### REFERENCE TO AN ELECTRONIC SEQUENCE LISTING

The contents of the electronic sequence listing (U0120.70155US01-KZM-SEQ.txt; Size: 4,423 bytes; and Date of Creation: Aug. 21, 2023) is herein incorporated by reference in its entirety.

### BACKGROUND

AAV mediated gene therapy is capable of delivering DNA directed transcription of epigenetic modulators as a modality to modulate offending endogenous genes. It is imperative that a clinically compatible system for endogenous gene regulation be developed. Historically gene regulation with rAAV vectors has mostly relied on the use of the tet operon, a bacterial based system responding to the presence of doxycycline as the small molecule needed for gene regulation. This promoter system has proven effective for gene regulation both as the “tet on” and “tet off” versions. However, the largest roadblock to this system is its reliance on the expression of foreign proteins of bacterial origin, which has been shown to be targeted for elimination by the immune system.

### SUMMARY

The present disclosure, at least in part, relates to compositions and methods for epigenetic modulating endogenous gene expression from vectors (e.g., viral vectors) in a cell or subject. In some embodiments, the vectors comprise expression constructs encoding comprising a promoter operably linked to a transgene, wherein the transgene encodes a FKBP-rapamycin binding protein (FRB), an epigenetic modulator, a rapamycin-binding protein (FKBP), and a DNA binding protein that specifically binds to an endogenous gene promoter. The disclosure is based, in part, epigenetic modulation of endogenous gene expression in a cell by varying a concentration of rapamycin or a rapalog in a cell containing such an expression vector.

In some aspects, the present disclosure provides an isolated nucleic acid comprising an expression cassette comprising a promoter operably linked to a transgene, wherein the transgene encodes a FKBP-rapamycin binding protein (FRB), an epigenetic modulator, a rapamycin-binding protein (FKBP), and a DNA binding protein that specifically binds to an endogenous gene promoter.

In some embodiments, the promoter is a constitutive promoter, an inducible promoter, or a tissue specific promoter. In some embodiments, the promoter is a minimal promoter. In some embodiments, the promoter is a chicken beta-actin promoter, a Human cytomegalovirus (CMV) promoter, or a chimeric CMV-chicken β-actin (CBA) promoter.

In some embodiments, the FKBP is FKBP12. In some embodiments, the DNA binding domain is a zinc finger domain, or dCas protein. In some embodiments, the DNA binding domain is a zinc finger domain. In some embodiments, the FKBP is directly fused to the DNA binding domain. In some embodiments, the FKBP is fused to the DNA binding domain via a linker. In some embodiments, the linker is a polypeptide linker. In some embodiments, the FKBP-DNA binding domain fusion protein is a FKBP12-zinc finger domain fusion protein. In some embodiments, the FKBP12-zinc finger domain fusion protein comprises an amino acid sequence at least 80% identical to amino acid sequence of SEQ ID NO: 1.

In some embodiments, the FRB domain is a FKBP12-rapamycin-associated protein (FRAP) domain. In some embodiments, the epigenetic modulator is a histone demethylases, a histone methyltransferases, a histone deacetylases, a histone acetyltransferases, a bromodomain-containing proteins, a kinase, or an actin-dependent regulators of chromatin. In some embodiments, the FRB domain is directly fused to the epigenetic modulator. In some embodiments, the epigenetic modulator is a histone demethylase. In some embodiments, the FRB is fused to the epigenetic modulator via a linker. In some embodiments, the linker is a polypeptide linker. In some embodiments, the isolated nucleic acid further comprises a IRES or a 2A peptide coding sequence. In some embodiments, the IRES or 2A is located between the FKBP12-zinc finger protein fusion protein and the FRB-epigenetic modulator fusion protein.

In some embodiments, the transgene each further comprises a 3′ untranslated region (3′UTR). In some embodiments, the transgene further comprises one or more miRNA binding sites. In some embodiments, the one or more miRNA binding sites are positioned in a 3′UTR of the transgene. In some embodiments, the at least one miRNA binding site is an immune cell-associated miRNA binding site or a liver-cell associated miRNA binding site. In some embodiments, the immune cell-associated miRNA is selected from: miR-106, miR-125a, miR-125b, miR-126a, miR-142, miR-146a, miR-15, miR-150, miR-155, miR-16, miR-17, miR-18, miR-181a, miR-19a, miR-19b, miR-20, miR-21a, miR-223, miR-24-3p, miR-29a, miR-29b, miR-29c, miR-302a-3p, miR-30b, miR-33-5p, miR-34a, miR-424, miR-652-3p, miR-652-5p, miR-9-3p, miR-9-5p, miR-92a, and miR-99b-5p. In some embodiments, the liver cell-associated miRNA is miR-122.

In some embodiments, the endogenous gene is LAMA1.

In some embodiments, the isolated nucleic acid further comprises two flanking long terminal repeats (LTRs).

In some embodiments, the isolated nucleic acid further comprising two flanking adeno-associated virus inverted terminal repeats (ITRs). In some embodiments, the ITRs are adeno-associated virus ITRs of a serotype selected from the group consisting of AAV1 ITR, AAV2 ITR, AAV3 ITR, AAV4 ITR, AAV5 ITR, and AAV6 ITR.

In some embodiments, the isolated nucleic acid is a closed-ended linear duplex DNA (ceDNA).

In some aspects, the present disclosure also provides a vector comprising the isolated nucleic acid as described herein. In some embodiments, the vector is a plasmid, a lentiviral vector, a retroviral vector, an anellovirus vector, or an adeno-associated virus (AAV) vector.

In some aspects, the present disclosure also provides a recombinant adeno-associated virus (rAAV) vector comprising, in 5′ to 3′ order: (a) a 5′ AAV ITR; (b) a promoter; (c) a transgene comprising, in 5′ to 3′ order: (A) a nucleotide sequence encoding a FRB-epigenetic modulator fusion protein; (B) a nucleotide sequence encoding an internal ribosome entry site (IRES); and (C) a nucleotide sequence encoding a FKBP-zinc finger domain fusion protein; and (d) a 3′ AAV ITR. In some aspects, the present disclosure also provides a recombinant adeno-associated virus (rAAV) vector comprising, in 5′ to 3′ order: (a) a 5′ AAV ITR; (b) a promoter; (c) a transgene comprising, in 3′ to 5′ order: (A) a nucleotide sequence encoding a FRB-epigenetic modulator fusion protein; (B) a nucleotide sequence encoding an internal ribosome entry site (IRES); and (C) a nucleotide sequence encoding a FKBP-zinc finger domain fusion protein; and (d) a 3′ AAV ITR. In some aspects, the present disclosure also provides a recombinant adeno-associated virus (rAAV) vector comprising, in 5′ to 3′ order: (a) a 5′ AAV ITR; (b) a promoter; (c) a transgene comprising, in 3′ to 5′ order: (A) a nucleotide sequence encoding a FRB-epigenetic modulator fusion protein; (B) a nucleotide sequence encoding an internal ribosome entry site (IRES); and (C) a nucleotide sequence encoding a FKBP-zinc finger domain fusion protein; and (d) a 3′ AAV ITR. In some aspects, the present disclosure also provides a recombinant adeno-associated virus (rAAV) vector comprising, in 5′ to 3′ order: (a) a 5′ AAV ITR; (b) a promoter; (c) a transgene comprising, in 3′ to 5′ order: (A) a nucleotide sequence encoding a FRB-epigenetic modulator fusion protein; (B) a nucleotide sequence encoding an internal ribosome entry site (IRES); and (C) a nucleotide sequence encoding a FKBP-zinc finger domain fusion protein; and (d) a 3′ AAV ITR.

In some aspects, the present disclosure also provides a recombinant lentivirus comprising a lentiviral capsid containing the isolated nucleic acid as described herein.

In some aspects, the present disclosure also provides a recombinant adeno-associated virus (rAAV) comprising: (i) an isolated nucleic acid as described herein; and (ii) at least one AAV capsid protein. In some embodiments, the capsid protein is of a serotype selected from AAV1, AAV2, AAV3, AAV4, AAV5, AAV6, AAV7, AAV8, AAV9 and a variant of any of the foregoing. In some embodiments, the rAAV is a single-stranded AAV (ssAAV).

In some aspects, the present disclosure also provides a recombinant adeno-associated virus (rAAV), comprising: (i) an AAV capsid protein; and (ii) an isolated nucleic acid comprising, in 5′ to 3′ order: (a) a 5′ AAV ITR; (b) a promoter; (c) a transgene comprising, in 5′ to 3′ order: (A) a nucleotide sequence encoding a FRB-epigenetic modulator fusion protein; (B) a nucleotide sequence encoding an internal ribosome entry site (IRES); and (C) a nucleotide sequence encoding a FKBP-zinc finger domain fusion protein; and (d) a 3′ AAV ITR.

In some aspects, the present disclosure also provides a host cell comprising: (i) isolated nucleic acid, the vector, the recombinant lentivirus, or the rAAV as described herein; and (ii) rapamycin or a rapalog. In some embodiments, the host cell is a mammalian cell, yeast cell, bacterial cell, or insect cell. In some embodiments, the rapalog is Sirolimus, Temsirolimus, or Everolimus. In some embodiments, the concentration of the rapalog is 10 nM to 2000 nM.

In some aspects, the present disclosure also provides a pharmaceutical composition comprising the isolated nucleic acid, the vector, the recombinant lentivirus o, the rAAV, or the host cell as described herein. In some embodiments, the pharmaceutical composition further comprises a pharmaceutically acceptable carrier. In some embodiments, the pharmaceutical composition is formulated for intravenous injection, intraperitoneal injection, intracranial injection, intratumoral injection, intramuscular injection, or intravitreal injection.

In some aspects, the present disclosure also provides a method for treating a disease in a subject in need thereof, the method comprising: (i) administering to the subject a therapeutically effective amount of the isolated nucleic acid, the vector, the recombinant lentivirus, the rAAV, the host cell, or the pharmaceutical composition as described herein; and (ii) administering to the subject an effective amount of rapamycin or a rapalog.

In some aspects, the present disclosure also provides a method for treating Congenital Muscular Dystrophy type 1A (MDC1A) in a subject in need thereof, the method comprising: (i) administering to the subject a therapeutically effective amount of the isolated nucleic acid, the vector, the recombinant lentivirus, the rAAV, the host cell, or the pharmaceutical composition as described herein; and (ii) administering to the subject an effective amount of rapamycin or a rapalog. In some embodiments, the zinc finger domain is capable of binding to endogenous LAMA1 promoter. In some embodiments, the subject is a non-human mammal. In some embodiments, the non-human mammal is mouse, rat, cat, dog, sheep, rabbit, horse, cow, goat, pig, guinea pig, hamster, chicken, turkey, or a non-human primate. In some embodiments, the subject is a human.

In some embodiments, the administration of (i) or (ii) is intravenous injection, intraperitoneal injection, intracranial injection, intratumoral injection, intramuscular injection, or intravitreal injection. In some embodiments, administering (i) and (ii) is concurrent. In some embodiments, administering (i) and (ii) is sequential. In some embodiments, administering (i) and (ii) is at different frequencies. In some embodiments, (i) is administered once and (ii) is administered repeatedly. In some embodiments, (ii) is administered every week, every two weeks, or every month.

In some embodiments, the dose of rapamycin or rapalog does not induce immunosuppression in the subject.

In some aspects, the present disclosure also provides a method for modulating endogenous expression in a subject, the method comprising: (i) administering the isolated nucleic acid, the vector, the recombinant lentivirus, the rAAV, the host cell, or the pharmaceutical composition as described herein, and a rapalog to a subject; (ii) measuring expression of transgene in the subject relative to a control expression level of the endogenous gene; (iii) adjusting the dose of rapalog-based on expression level measured in (ii), wherein if the expression level measured in (ii) is increased relative to the control level, administering the same or less concentration of the rapalog; and wherein if the expression level measured in (ii) is the same or decreased relative to the control level, administering a higher concentration of the rapalog to the subject.

### BRIEF DESCRIPTION OF DRAWINGS

FIGS. 1A-1B are schematic designs of a rapalog regulatable system. FIG. 2A shows the rapalog-regulatable system which comprises two Rapa binding domains (FKBP and FRB). The FRP domain is fused to an epigenetic modulator which can alter the chromatin structure of an endogenous gene. The FKBP domain is fused to a zinc finger nuclease engineered to specifically bind the endogenous gene to be regulated. In the presence of ligand the domains are brought together as a functional epigenetic modulation unit at the endogenous gene promoter sequence. FIG. 2B are graphs showing how the rapalog regulatable system behave in the cells in the absence (top panel) or presence (bottom panel) of the heterodimerizer (e.g., rapalog). In these systems, the small molecule or “dimerizer” causes heterodimerization of two proteins comprising an epigenetic modulator and a DNA binding domain. The heterodimerization of these proteins leads to alteration of expression of an endogenous gene. Top panel shows when no addition of A/C Heterodimerizer (−), transcription is not induced. Bottom panel shows the addition of A/C Heterodimerizer (+) leads to transcription.

### DETAILED DESCRIPTION

Aspects of the disclosure relate to compositions and methods for epigenetic modulating endogenous gene expression from vectors (e.g., viral vectors) in a cell or subject. In some embodiments, the vectors comprise expression constructs encoding comprising a promoter operably linked to a transgene, wherein the transgene encodes a FKBP-rapamycin binding protein (FRB), an epigenetic modulator, a rapamycin-binding protein (FKBP), and a DNA binding protein that specifically binds to an endogenous gene promoter. The disclosure is based, in part, modulation of endogenous gene expression in a cell by varying a concentration of rapamycin or a rapalog in a cell containing such an expression vector.

### Regulatable Transgene Constructs

The disclosure relates, in some aspects, to isolated nucleic acids comprising an expression cassette encoding one or more transgenes. A “nucleic acid” sequence refers to a DNA or RNA sequence. In some embodiments, proteins and nucleic acids of the disclosure are isolated. As used herein, the term “isolated” means artificially produced. As used herein, with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.). In some embodiments, a nucleic acid encoding a complement control protein or portion thereof is codon-optimized (e.g., codon-optimized for expression in human cells).

In some embodiments, an isolated nucleic acid comprises nucleic acid sequences encoding protein domains that can be activated by the presence of rapamycin or a rapalog.

Rapamycin, also referred to as sirolimus, is a macrolide compound produced by the bacteria Streptomyces hygroscopicus, which functions as an immunosuppressant and antifungal agent. In mammals, rapamycin binds to the mammalian target of rapamycin (mTOR), a serine/threonine-specific protein kinase that regulates cellular metabolism, growth, and proliferation. In some embodiments, rapamycin is used in combination with fusion constructs containing a rapamycin-binding FRB domain and an FKBP domain in order to mediate chemically-induced dimerization of the constructs, resulting in an inducible gene expression system. Such a system is described, for example, by Rivera et al. (1996) Nature Medicine. 2 (9): 1028-32. In some embodiments, a regulatable promoter is activated by (e.g., binds to) rapamycin or a rapalog (a derivative of rapamycin). Examples of rapalogs include but are not limited to temsirolimus, everolimus, and ridaforolimus. In some embodiments, a regulatable promoter is activated by (e.g., binds to) an ATP-competitive mTOR kinase inhibitor (e.g., a second generation mTOR inhibitor).

In some embodiments, the isolated nucleic acid comprises a expression cassette, which comprises a promoter operably linked to a transgene. In some embodiments, an expression cassette comprises a promoter operably linked to a transgene, wherein the transgene encodes a FKBP-rapamycin binding (FRB), an epigenetic modulator, a rapamycin-binding protein (FKBP), and a DNA binding protein that specifically binds to a endogenous gene promoter.

In some embodiments, a promoter is a constitutive promoter, inducible promoter, or a tissue-specific promoter.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the chimeric cytomegalovirus chimeric cytomegalovirus (CMV)/Chicken β-actin (CB) promoter (CBA promotor), the SV40 promoter, the dihydrofolate reductase promoter, the β-actin promoter, the phosphoglycerol kinase (PGK) promoter, and the EF1α promoter [Invitrogen]. In some embodiments, a promoter is an RNA pol II promoter. In some embodiments, a promoter is the chimeric cytomegalovirus chimeric cytomegalovirus (CMV)/Chicken β-actin (CB) promoter (CBA promoter). In some embodiments, a promoter is an RNA pol III promoter, such as U6 or H1.

Examples of inducible promoters regulated by exogenously supplied signals include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: retinoschisin proximal promoter, interphotoreceptor retinoid-binding protein enhancer (RS/IRBPa), rhodopsin kinase (RK), liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (α-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific VGF gene promoter (Piccioli et al., Neuron, 15:373-84 (1995)), among others which will be apparent to the skilled artisan.

In some embodiments, a promoter is a chicken beta-actin (CB) promoter. A chicken beta-actin promoter may be a short chicken beta-actin promoter or a long chicken beta-actin promoter. In some embodiments, a promoter (e.g., a chicken beta-actin promoter) comprises an enhancer sequence, for example a cytomegalovirus (CMV) enhancer sequence. A CMV enhancer sequence may be a short CMV enhancer sequence or a long CMV enhancer sequence. In some embodiments, a promoter comprises a long CMV enhancer sequence and a long chicken beta-actin promoter. In some embodiments, a promoter comprises a short CMV enhancer sequence and a short chicken beta-actin promoter. However, the skilled artisan recognizes that a short CMV enhancer may be used with a long CB promoter, and a long CMV enhancer may be used with a short CB promoter (and vice versa).

Aspects of the disclosure relate to expression constructs encoding a fusion protein that comprises a rapamycin-binding protein (FKBP). A rapamycin-binding protein (FKBP), as used herein, refers to a cytosolic receptor for rapamycin or rapalog and is highly conserved across species lines. Generally, FKBPs are proteins or protein domains which are capable of binding to rapamycin or to a rapalog and further forming a tripartite complex with an FRB-containing protein. Information concerning the nucleotide sequences, cloning, and other aspects of various FKBP species is known in the art, see e.g., Staendart et al, 1990, Nature 346, 671-674 (human FKBP12); Kay, 1996, Biochem. J. 314, 361-385 (review). Homologous FKBP proteins in other mammalian species, in yeast, and in other organisms are also known in the art and may be used in the fusion proteins disclosed herein. See e.g. Kay, 1996, Biochem. J. 314, 361-385 (review). An FKBP peptide is capable of binding to rapamycin or a rapalog and participating in a tripartite complex with a FRB-containing protein. Non-limiting examples of FKBR include naturally occurring peptide sequence derived from the human FKBP12 protein, a peptide sequence derived from another human FKBP, from a murine or other mammalian FKBP, or from some other animal, yeast or fungal FKBP. In some embodiments, the FKBP is FKBP12.

Aspects of the disclosure relate to expression constructs encoding a fusion protein that comprises a FKBP-rapamycin binding (FRB) domain. An FKBP-rapamycin binding (FRB) domain, as used herein, refers to polypeptide which are capable of forming a tripartite complex with an rapamycin-binding protein (FKBP) and rapamycin (or a rapalog). FRB domains are present in a number of naturally occurring proteins. Non-limiting examples of an FRB domain include FRAP proteins from human and other species, yeast proteins including Tor1 and Tor2, or a Candida FRAP homolog. Information concerning the nucleotide sequences, cloning, and other aspects of these proteins is already known in the art. For example, protein source reference/sequence accession numbers human FRAP Brown et al, 1994, Nature 369, 756-758; GenBank accession #L34075, NCBI Seq ID 508481; Chiu et al, 1994, PNAS USA 91, 12574 12578; Chen et al, 1995, PNAS USA 92, 4947 4951 murine RAPT1 Chiu et al, supra. yeast Tor1 Helliwell et al, 1994, Mol Cell Biol 5, 105-118; EMBL Accession #X74857 NCBI Se Id #468738 yeast Tor2 Kunz et al, 1993, Cell 73, 585-596; EMBL Accession #X71416, NCBI Seq ID 298027 Candida W095/33052 homolog. In some embodiments, the FRB is FRAP.

In some embodiments, a fusion protein comprises a FKBP or FRB and a DNA binding domain (DBD). A DNA binding domain, as used herein, refers to proteins domains that have DNA-binding capacity and thus have a specific or general affinity for single- or double-stranded DNA. Non-limiting examples of DNA binding domain include transcription factors, polymerases, histones, nucleases (e.g., zinc finger nucleases (ZFNs), transcription activator-like effector nucleases (TALENs), meganucleases (e.g., engineered meganucleases) and CRISPR-associated proteins (Cas nucleases)). In some embodiments, the DNA binding domain binds to the promoter of an endogenous gene.

In some embodiments, a DNA binding protein is a zinc finger domain. A zinc finger domain, as used herein, refers to small protein motifs that contain multiple finger-like protrusions that make tandem contacts with their target molecule. Zinc finger domains are capable of binding bind DNA, RNA, protein, and/or lipid substrates. The binding of a zinc-finger domain to its target site juxtaposes three base pairs on DNA to a few amino acids in the α-helix structure. The identity of the amino acids at the contact site defines the DNA sequence recognition specificity of zinc fingers. Thus, by changing these amino acids, a high degree of selectivity can be achieved toward a given three base-pair DNA sequence (see, e.g., Cassandri, Zinc-finger proteins in health and disease, Cell Death Discovery volume 3, Article number: 17071 (2017)). In some embodiments, the zinc finger protein binds to DNA sequences. In some embodiments, the zinc finger domain binds to the promoter of an endogenous gene. In some embodiments, the zinc finger domain is designed based on the endogenous gene of interest.

In some embodiments, a DNA binding domain is a CRISPR-associated protein. The term “CRISPR” refers to “clustered regularly interspaced short palindromic repeats”, which are DNA loci containing short repetitions of base sequences. CRISPR loci form a portion of a prokaryotic adaptive immune system that confers resistance to foreign genetic material. Each CRISPR loci is flanked by short segments of “spacer DNA”, which are derived from viral genomic material. In the Type II CRISPR system, spacer DNA hybridizes to transactivating RNA (tracrRNA) and is processed into CRISPR-RNA (crRNA) and subsequently associates with CRISPR-associated proteins (Cas proteins) to form complexes that recognize and degrade foreign DNA. Non-limiting Examples of Cas proteins include, but are not limited to Cas9, dCas9, Cas6, Cas13, CasRX, Cpf1, and variants thereof. In some embodiments, the Cas protein is a dCas protein. A dCas protein, as used herein, refers to a Cas protein is modified (e.g. genetically engineered) to lack nuclease activity. For example, “dead” Cas9 (dCas9) protein binds to a target locus but does not cleave said locus. Non-limiting examples for dCas protein include dCas9, and dCas13.

In some aspects, the disclosure relates to expression constructs encoding one or more epigenetic modulators. As used herein, the term “epigenetic modulator” refers to an agent that alters the transcriptional activity of a gene (e.g., an endogenous gene), e.g., by affecting the chromatin state of that gene (e.g., promoting a condensed chromatin state to inhibit transcription or a relaxed chromatin state to enhance transcription). In some embodiments, an epigenetic modulator is an agent that, when delivered to a cell, modulates transcription of an endogenous gene in the cell. In some embodiments, an epigenetic modulator directly modulates an endogenous gene expression. In some embodiments, an epigenetic modulator indirectly modulates an endogenous gene expression (e.g., does not bind directly to the promoter region of the endogenous gene, or regulates expression or activity of a direct chromatin modifier of the endogenous gene expression). For example, in some embodiments an epigenetic modulator is an RNAi molecule or small molecule inhibitor that targets a regulator (e.g., a chromatin modifier that modulates chromatin state at an endogenous locus) of an endogenous gene expression, and thereby functions as an epigenetic modulator of endogenous gene expression.

The chromatin state (e.g., packaging of DNA with histone and non-histone proteins) of a cell has significant effects on gene expression. In some embodiments, the disclosure relates to chromatin modifiers (e.g., chromatin remodeling proteins and genes encoding the same) that are capable of modulating expression of an endogenous gene in cells. In some embodiments, an epigenetic modulator of an endogenous gene is a chromatin modifier. As used herein, the term “chromatin modifier” refers to an agent (e.g., an enzyme or transcription factor) that modifies DNA (e.g., by methylation) or post-translationally modifies histone proteins (for example by phosphorylation, acetylation, methylation or ubiquitination), resulting in alteration of chromatin structure and thus modified gene expression. Examples of chromatin modifiers include, but are not limited to histone demethylases (e.g., lysine demethylase enzymes), histone methyltransferases, histone deacetylases, histone acetyltransferases, certain bromodomain-containing proteins, kinases (e.g., kinases that phosphorylate histones), and actin-dependent regulators of chromatin. In some embodiments, one or more chromatin remodeling proteins is present in a chromatin structure remodeling complex (RSC). Thus, in some embodiments, a chromatin modifier of an endogenous gene is a component (e.g., a protein present in) an RSC.

As used herein, the term “histone demethylase” refers to an enzyme that catalyzes the removal of a methyl group from a histone protein. In some embodiments, histone demethylase enzymes comprise one or more of the following domains: Swi3, Rsc and Moira (SWIRM1) domain, Jumonji N- or C-terminal (JmjN or JmjC) domain, PHD-finger domain, Zinc-finger domain, amine oxidase domain, and Tudor domain. For example, in some embodiments, a histone deacetylase comprises at least one Tudor domain and two Jmj domains (e.g. one JmjN domain and one JmjC domain). In some embodiments, a histone demethylase is a lysine-specific histone demethylase. Non-limiting examples of lysine-specific histone demethylases include KDM4A, KDM4B, KDM4C, KDM4D, KDM6A, KDM6B, and PHF2. In some embodiments, an epigenetic modulator of an endogenous gene is a histone demethylase inhibitor.

As used herein, the term “bromodomain-containing protein” refers to a protein that has a bromodomain that recognizes monoacetylated lysine residues. Generally, bromodomain-containing (BRD) proteins bind to acetylated histones and, in some embodiments, mediate chromatin remodeling. In some embodiments, BRD proteins comprise at least one bromodomain (e.g., 1, 2, 3, 4, 5, or more bromodomains) and an Extra-Terminal (ET) domain. Non-limiting examples of BRD proteins include BRD2, BRD3, BRD4, BRDT, BRPF1, BRPF3, BPTF, BAZ1A, BAZ1B, and BAZ2A. In some embodiments, an epigenetic modulator of an endogenous gene is a BRD protein.

As used herein, the term “actin-dependent regulator of chromatin” refers to a protein that is a member of the SWI/SNF Related Matrix Associated Actin Dependent Regulator of Chromatin family. Generally, members of the SWI/SNF protein family comprise a helicase domain and am ATPase domain and function to regulate transcription of certain genes by altering chromatin structure. In some embodiments, an actin-dependent regulator of chromatin further comprises one or more Swi3, Ada2, N-Cor, and TFIIIB (SANT) domains. In some embodiments, one or more actin-dependent regulators of chromatin are present in the chromatin remodeling and splicing factor (RSF) complex. Non-limiting examples of actin-dependent regulators of chromatin include SMARCA5, SMARCB 1, SMARCA4, SMARCC1, SMARCC2, SMARCD1, SMARCD2, and SMARCD3. In some embodiments, an epigenetic modulator of an endogenous gene is an actin-dependent regulator of chromatin.

As used herein, the term “histone methyltransferase” refers to an enzyme that catalyzes the transfer of at least one methyl group (e.g., 1, 2, 3, or more methyl groups) to lysine and/or arginine residues of a histone protein. Generally, histone methyltransferase enzymes are characterized as either lysine-specific or arginine-specific methyltransferases.

In some embodiments, an epigenetic modulator of an endogenous gene is a lysine-specific methyltransferase. There are two families of lysine-specific methyltransferases: SET domain-containing methyltransferases and non-SET domain-containing methyltransferases. Examples of lysine-specific histone methyltransferases include, but are not limited to, ASH1L, DOT1L, EHMT1, EHMT2, EZH1, EZH2, MLL, MLL2, MLL3, MLL4, MLL5, NSD1, PRDM1, KMT2A, KMT2C, KMT2E, SET, SETBP1, SETD1A, SETD2, SETD3, SETD4, SETD5, SETD6, SETD7, SETD9, SETD1B, SMYD1, SMYD2, SMYD3, SMYD4, SMYD5, SUV39H1, SUV39H2, SUV420H1, and SUV420H2. In some embodiments, an epigenetic modulator of an endogenous gene is a lysine-specific methyltransferase.

In some embodiments, an epigenetic modulator of an endogenous gene is an arginine-specific methyltransferase. Arginine-specific methyltransferases, also referred to as PRMTs, are generally classified into two groups. One group of PRMTs comprising PRMT1, PRMT3, CARM1, PRMT4, and Rmt1/Hmt1 produce monomethylarginine and asymmetric dimethylarginine residues. A second group of PRMTs comprising JBP1 and PRMT5 produces monomethyl or symmetric dimethylarginine residues. In some embodiments, an epigenetic modulator of an endogenous gene is an arginine-specific methyltransferase.

In some embodiments, an epigenetic modulator of an endogenous gene is a kinase (e.g. a serine-threonine kinase, for example NEK6), deacetylase enzyme (e.g., a histone deacetylase, for example HDAC1), splicing factor protein (e.g., a member of the splicing factor 3b protein complex, for example SF3B1), polymerase (e.g., PARP1, PARP2, PARP3, etc.), ligase (e.g., UFL1), hydrolase (e.g., BAP1), peptidase (e.g., a ubiquitin specific peptidase, for example USP3, USP7, USP16, USP21, or USP22), or a protease (e.g., a histone deubiquitinase, for example MYSM1). In some embodiments, an epigenetic modulator of an endogenous gene is any one of the foregoing proteins (e.g., a kinase, a deacetylase enzyme, a splicing factor protein, a polymerase, a ligase i, a hydrolase, a peptidase, or a protease).

In some embodiments, the transgene in the expression cassettes encodes fusion proteins. A fusion protein, as used herein, refer to proteins comprises various component domains or sequences that are mutually heterologous in the sense that they do not occur together in the same arrangement in nature. More specifically, the component portions are not found in the same continuous polypeptide or nucleotide sequence or molecule in nature, at least not in the same order or orientation or with the same spacing present in the fusion protein as described herein.

In some embodiments, fusion proteins described herein contain at least one “receptor” or “ligand binding” domain comprising peptide sequence derived from a FKBP protein and at least one other domain, heterologous with respect to the receptor domain, but capable, upon oligomerization of the chimeric protein molecules, of triggering a cellular or biological response (e.g., binding to the promoter of an endogenous gene). In some embodiments, the other domains comprise a DNA-binding domain (e.g., zinc finger domain) and a epigenetic modulator (e.g., a histone demethylase), paired such that oligomerization of the fusion proteins represents assembly of a transcription factor complex which triggers transcription of an endogenous gene linked to a DNA sequence recognized by (capable of specific binding interaction with) the DNA binding domain (e.g., the promoter of the endogenous gene).

In some embodiments, a FRB domain is fused to a epigenetic modulator. In some embodiments, a FRB domain is fused to a DNA binding domain. In some embodiments, a FKBP is fused to a DNA binding domain. In some embodiments, a FRB domain is fused to a DNA binding domain, and a FKBP is fused to a epigenetic modulator. In some embodiments, a FKBP is fused to an epigenetic modulator. In some embodiments, the FRB domain is fused to a epigenetic modulator, and the FKBP is fused to a DNA binding domain. In some embodiments, a FRAP is fused to an epigenetic modulator, and a FKBP12 is fused to a zinc finger domain.

In some embodiments, domains of a fusion protein are connected without a linker. In some embodiments, the domains of a fusion protein are connected via a linker. Suitable linkers are known in the art. (See, e.g., Chen et al., Fusion protein linkers: property, design and functionality, Adv Drug Deliv Rev. 2013 October; 65(10):1357-69). In some embodiments, an FKBP-DNA binding domain fusion protein comprises an amino acid sequence at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to amino acid sequence of SEQ ID NO: 1.

An exemplary amino acid sequence for FKBP12-zinc finger domain fusion protein is set forth in SEQ ID NO: 1.

MDYPAAKRVKLDSRERPYACPVESCDRRFSRSDELTRHIRIHTGQKPFQ
CRICMRNFSRSDHLTTHIRTHTGGGRRRKKRTSIETNIRVALEKSFLEN
QKPTSEEITMIADQLNMEKEVIRVWFCNRRQKEKRINTRGVQVETISPG
DGRTFPKRGQTCVVHYTGMLEDGKKFDSSRDRNKPFKFMLGKQEVIRGW
EEGVAQMSVGQRAKLTISPDYAYGATGHPGIIPPHATLVFDVELLKLEV
EGVQVETISPGDGRTFPKRGQTCVVHYTGMLEDGKKFDSSRDRNKPFKF
MLGKQEVIRGWEEGVAQMSVGQRAKLTISPDYAYGATGHPGIIPPHATL
VFDVELLKLETRGVQVETISPGDGRIFPKRGQTCVVHYTGMLEDGKKFD
SSRDRNKPFKFMLGKQEVIRGWEEGVAQMSVGQRAKLTISPDYAYGATG
HPGIIPPHATLVFDVELLKLETSY

In some embodiments, a transgene encodes a FKBP-DNA binding domain fusion protein (e.g., FKBP12-zinc finger protein fusion protein) and a FRB-epigenetic modulator fusion protein (e.g., FRAP-histone demethylase fusion protein). A transgene described herein is capable of form a heterodimer in the presence of rapamycin or rapalogs. In some embodiments, the DNA binding domain of the heterodimer is designed to recognize an endogenous gene promoter, and the epigenetic modulator is capable of modulating the expression of the endogenous gene driven by the endogenous gene promoter. An endogenous gene promoter, as used herein, refers to a native promoter that drives the expression of a gene that's naturally expressed by a subject. In some embodiments, an endogenous gene is expressed in all developmental stage of a subject. In some embodiments, an endogenous gene is expressed in a certain developmental stage of a subject. Aberrant expression of an endogenous gene (e.g., by genetic mutation or epigenetic alteration) is associated with many diseases. Non-limiting examples of endogenous genes and their respective promoters include Laminin Subunit Alpha 1 (LAMA1) and LAMA1 promoter, Glyceraldehyde 3-phosphate dehydrogenase (GAPDH) gene and GAPDH gene promoter, a Macrophage Migration Inhibitory Factor (MIF) gene and MIF gene promoter, a Small Nuclear Ribonucleoprotein D2 Polypeptide (SNRPD2) gene and SNRPD2 promoter, Non-POU Domain-Containing Octamer-Binding Protein (NONO) and NONO promoter, phosphoglycerate kinase 1 (PGK1) and PGK1 promoter, or Peptidylprolyl Isomerase H (PPIH) and PPIH promoter, an Eukaryotic Translation Elongation Factor 1 Alpha 1 (EEF1A1) gene and EEF1A1 promoter, ubiquitin E3 ligase (UBE3A) gene and UBE3A promoter, Insulin-like growth factor 2 (IGF2) gene and IGF2 promoter, cyclin-dependent kinase inhibitor (CDKNIC) gene and CDKNIC promoter, guanine nucleotide binding protein, alpha stimulating activity polypeptide 1 (GNAS1) gene and GNAS1 promoter, FMRP Translational Regulator 1 (FMR-1) gene and FMR-1 promoter, (Methyl-CpG Binding Protein 2) MeCP2 gene and MeCP2 promoter, Reelin (RELN) gene and RELN promoter, SRY-Box Transcription Factor 10 (SOX10) gene and SOX10 promoter, Glutamate Decarboxylase 1 (GAD1) gene and GAD1 promoter, WDR18 and WDR18 promoter, Peptidylprolyl Isomerase E Like Pseudogene (PPIEL) gene and PPIEL promoter, Ribosomal Protein L39 (RPL39) gene and RPL39 promoter, or Cryptochrome Circadian Regulator 1 (CRY1) gene and CRY1 promoter. Identification of diseases related endogenous genes and their respective promoters have been previously described, e.g., by Chowdhary et al., A Database of Annotated Promoters of Genes Associated with Common Respiratory and Related Diseases, Am J Respir Cell Mol Biol. 2012 July; 47(1):112-9; Klingenhoff et al., Linking disease-associated genes to regulatory networks via promoter organization, Nucleic Acids Res. 2005; 33(3): 864-872.

In some embodiments, a transgene encodes a FKBP-DNA binding domain fusion protein (e.g., FKBP12-zinc finger protein fusion protein) and a FRB-epigenetic modulator fusion protein (e.g., FRAP-histone demethylase fusion protein). In some embodiments, the expression cassette is a multicistronic expression cassette.

A multicistronic expression cassette, as used herein, refers to expression cassettes that simultaneously express two or more separate proteins from the same mRNA. In some embodiments, the expression cassette further comprises an IRES or a 2A self-cleaving peptide coding sequence. In some embodiments, the internal ribosome entry site (IRES) or the 2A peptide coding sequence is located between the FKBP-DNA binding domain fusion protein (e.g., FKBP12-zinc finger protein fusion protein) and a FRB-epigenetic modulator fusion protein (e.g., FRAP-histone demethylase fusion protein). An internal ribosome entry site (IRES), is an RNA element that allows for translation initiation in a cap-independent manner, as part of the greater process of protein synthesis. A 2A self-cleaving peptide, as used herein, refers to a class of 18-22 aa-long peptides, which can induce the cleaving of the recombinant protein in cell. In some embodiments, the transgene comprises an IRES located between the FKBP-DNA binding domain fusion protein (e.g., FKBP12-zinc finger protein fusion protein) and a FRB-epigenetic modulator fusion protein (e.g., FRAP-histone demethylase fusion protein).

In some embodiments, the FKBP-DNA binding domain fusion protein (e.g., FKBP12-zinc finger protein fusion protein) and a FRB-epigenetic modulator fusion protein (e.g., FRAP-histone demethylase fusion protein) forms a heterodimer in response to rapamycin or rapalog. Rapamycin was initially discovered as an antifungal metabolite produced by Streptomyces hygroscopicus from a soil sample of Easter Island (also known as Rapa Nui). Subsequently, rapamycin was found to possess immunosuppressive and anti-proliferative properties in mammalian cells, spurring an interest in identifying the mode of action of rapamycin. Rapamycin was shown to be a potent inhibitor of S6K1 activation, a serine/threonine kinase activated by a variety of agonists (Chung et al., 1992; Kuo et al., 1992; Price et al., 1992) and an important mediator of PI3 kinase signaling. Concurrently, the target of rapamycin (TOR) was identified in yeast and animal cells (Laplante and Sabatini, 2012; Loewith and Hall, 2011). Rapamycin functions by binding with high affinity to FKBP, and then to the large PI3K homolog FRAP (RAFT, mTOR), thereby acting as a heterodimerizer to join the two proteins together. In some embodiments, a rapalogs is a compound other than rapamycin, preferably of molecular weight below 5 kD, more preferably below 2.5 kD, which is capable of binding with an FKBP fusion protein and of forming a complex with an FKBP fusion protein and an FRB fusion protein. Rapalogs are rapamycin analogs alco capable of inhibiting mTOR signaling. Rapalogs have been previously described, e.g., Abdel-Magid et al., Rapalogs Potential as Practical Alternatives to Rapamycin, ACS Medicinal Chemistry Letters 2019 10 (6), 843-845. In some embodiments, the rapalog is Sirolimus, Temsirolimus, or Everolimus.

In some embodiments, a transgene encodes a FKBP-DNA binding domain fusion protein (e.g., FKBP12-zinc finger protein fusion protein) and a FRB-epigenetic modulator fusion protein (e.g., FRAP-histone demethylase fusion protein). The zinc finger protein is capable of binding to promoter of an endogenous gene, and the epigenetic modulator is capable of altering the chromatin structure of the endogenous gene. In some embodiments, in the absence of rapamycin or rapalog, the FKBP-DNA binding domain fusion protein (e.g., FKBP12-zinc finger protein fusion protein) does not form a heterodimer with a FRB-epigenetic modulator fusion protein (e.g., FRAP-histone demethylase fusion protein), thus the endogenous gene chromatin structure is not altered. In other embodiments, in the presence of rapamycin or rapalog, the FKBP-DNA binding domain fusion protein (e.g., FKBP12-zinc finger protein fusion protein) forms a heterodimer with a FRB-epigenetic modulator (e.g., FRAP-histone demethylase fusion protein), thus the promoter of an endogenous gene is bound by the DNA binding domain, and its chromatic structure is altered by the epigenetic modulator.

In some embodiments, the isolated nucleic acid described herein is a multicistronic expression construct. In some embodiments, multicistronic expression constructs comprises expression cassettes that are positioned in different ways. For example, in some embodiments, a multicistronic expression construct is provided in which a first expression cassette is positioned adjacent to a second expression cassette. In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1 - - - promoter 2/encoding sequence 2-3′,
>>>>>>>>>>>>>>>>>>>>>>> >>>>>>>>>>>>>>>>>>>>>>>
the expression cassettes are in the same orientation, the arrows indicate the direction of transcription of each of the cassettes. For another example, if a given nucleic acid construct comprises a sense strand comprising two expression cassettes in the configuration
5′-promoter 1/encoding sequence 1 - - - encoding sequence 2/promoter 2-3′,
>>>>>>>>>>>>>>>>>>>>>>> <<<<<<<<<<<<<<<<<<<<<
the expression cassettes are in opposite orientation to each other and, as indicated by the arrows, the direction of transcription of the expression cassettes, are opposed. In this example, the strand shown comprises the antisense strand of promoter 2 and encoding sequence 2.

For another example, if an expression cassette is comprised in an AAV construct, the cassette can either be in the same orientation as an AAV ITR, or in opposite orientation. AAV ITRs are directional. For example, the 3′ITR would be in the same orientation as the promoter 1/encoding sequence 1 expression cassette of the examples above, but in opposite orientation to the 5′ITR, if both ITRs and the expression cassette would be on the same nucleic acid strand.

In some embodiments, a multicistronic expression construct is provided that allows efficient expression of a first encoding nucleic acid sequence driven by a first promoter and of a second encoding nucleic acid sequence driven by a second promoter without the use of transcriptional insulator elements. Various configurations of such multicistronic expression constructs are provided herein, for example, expression constructs harboring a first expression cassette comprising an intron and a second expression cassette positioned within the intron, in either the same or opposite orientation as the first cassette. Other configurations are described in more detail elsewhere herein.

In some embodiments, multicistronic expression constructs are provided allowing for efficient expression of two or more encoding nucleic acid sequences. In some embodiments, the multicistronic expression construct comprises two expression cassettes. In some embodiments, a first expression cassette of a multicistronic expression construct as provided herein comprises a first RNA polymerase II promoter and a second expression cassette comprises a second RNA polymerase II promoter. In some embodiments, a first expression cassette of a multicistronic expression construct as provided herein comprises an RNA polymerase II promoter and a second expression cassette comprises an RNA polymerase III promoter.

In some embodiments, the transgene comprises a 3′-untranslated region (3′-UTR). In some embodiments, the disclosure relates to isolated nucleic acids comprising a transgene which comprises one or more miRNA binding sites. Without wishing to be bound by any particular theory, incorporation of miRNA binding sites into gene expression constructs allows for regulation of transgene expression (e.g., inhibition of transgene expression) in cells and tissues where the corresponding miRNA is expressed. In some embodiments, incorporation of one or more miRNA binding sites into a transgene allows for de-targeting of transgene expression in a cell-type specific manner. In some embodiments, one or more miRNA binding sites are positioned in the 3′ untranslated region (3′-UTR) of a transgene, for example between the last codon of a nucleic acid sequence encoding a transgene and a poly A sequence.

In some embodiments, a transgene comprises one or more (e.g., 1, 2, 3, 4, 5, or more) miRNA binding sites that de-target expression of the transgene (e.g., FRAP-zinc finger domain fusion protein and FRB-epigenetic modulator fusion protein) from immune cells (e.g., antigen presenting cells (APCs), such as macrophages, dendrites, etc). Incorporation of miRNA binding sites for immune-associated miRNAs may de-target transgene expression from antigen presenting cells and thus reduce or eliminate immune responses (cellular and/or humoral) produced in the subject against products of the transgene, for example as described in US 2018/0066279, the entire contents of which are incorporated herein by reference.

In some embodiments, the transgene comprises one or more (e.g., 1, 2, 3, 4, 5, or more) miRNA binding sites that de-target expression of the transgene from liver cells. For example, in some embodiments, a transgene comprises one or more miR-122 binding sites.

As used herein an “immune cell-associated miRNA” is a miRNA preferentially expressed in cells of the immune system, such as an antigen presenting cell (APC). In some embodiments, an immune cell-associated miRNA is an miRNA expressed in immune cells that exhibits at least a 2-fold, 3-fold, 4-fold, 5-fold, 6-fold, 7-fold, 8-fold, 9-fold, 10-fold higher level of expression in an immune cell compared with a non-immune cell (e.g., a control cell, such as a HeLa cell, HEK293 cell, mesenchymal cell, etc.). In some embodiments, the cell of the immune system (immune cell) in which the immune cell-associated miRNA is expressed is a B cell, T cell, Killer T cell, Helper T cell, γδ T cell, dendritic cell, macrophage, monocyte, vascular endothelial cell, or other immune cell. In some embodiments, the cell of the immune system is a B cell expressing one or more of the following markers: B220, BLAST-2 (EBVCS), Bu-1, CD19, CD20 (L26), CD22, CD24, CD27, CD57, CD72, CD79a, CD79b, CD86, chB6, D8/17, FMC7, L26, M17, MUM-1, Pax-5 (BSAP), and PC47H. In some embodiments, the cell of the immune system is a T cell expressing one or more of the following markers: ART2, CD1a, CD1d, CD11b (Mac-1), CD134 (OX40), CD150, CD2, CD25 (interleukin 2 receptor alpha), CD3, CD38, CD4, CD45RO, CD5, CD7, CD72, CD8, CRTAM, FOXP3, FT2, GPCA, HLA-DR, HML-1, HT23A, Leu-22, Ly-2, Ly-m22, MICG, MRC OX 8, MRC OX-22, OX40, PD-1 (Programmed death-1), RT6, TCR (T cell receptor), Thy-1 (CD90), and TSA-2 (Thymic shared Ag-2). In some embodiments, the immune cell-associated miRNA is selected from: miR-106, miR-125a, miR-125b, miR-126a, miR-142, miR-146a, miR-15, miR-150, miR-155, miR-16, miR-17, miR-18, miR-181a, miR-19a, miR-19b, miR-20, miR-21a, miR-223, miR-24-3p, miR-29a, miR-29b, miR-29c, miR-302a-3p, miR-30b, miR-33-5p, miR-34a, miR-424, miR-652-3p, miR-652-5p, miR-9-3p, miR-9-5p, miR-92a, and miR-99b-5p. In some embodiments, a transgene described herein comprises one or more binding sites for miR-142.

As used herein, a nucleic acid sequence (e.g., coding sequence) and regulatory sequences are said to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, a transgene described herein comprises a Kozak sequence. A

Kozak sequence is a nucleic acid motif comprising a consensus sequence that is found in eukaryotic mRNA and plays a role in initiation of protein translation. In some embodiments, the Kozak sequence is positioned between the intron and the transgene encoding transgene described herein

An isolated nucleic acid described by the disclosure may encode a transgene that further comprises a polyadenylation (poly A) sequence. In some embodiments, a transgene comprises a poly A sequence is a rabbit beta-globulin (RBG) poly A sequence.

In some embodiments, the isolated nucleic acid comprises inverted terminal repeats. The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, one or more transgene (e.g., the transgene described herein) and its regulatory sequences, and 5′ and 3′ AAV inverted terminal repeats (ITRs). The transgene may comprise a region encoding a transgene (e.g., FRAP-zinc finger domain fusion protein and FRB-epigenetic modulator fusion protein) and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid further comprises a region (e.g., a second region, a third region, a fourth region, etc.) comprising a second AAV ITR. In some embodiments, an isolated nucleic acid encoding a transgene is flanked by AAV ITRs (e.g., in the orientation 5′-ITR-transgene-ITR-3′). In some embodiments, the AAV ITRs are selected from the group consisting of AAV1 ITR, AAV2 ITR, AAV3 ITR, AAV4 ITR, AAV5 ITR, and AAV6 ITR.

In some embodiments, the isolated nucleic acid as described herein comprises a 5′ AAV ITR, an expression cassette described herein, and a 3′ AAV ITR.

Also within the scope of the present disclosure are plasmids comprising the isolated nucleic acid described herein. In some embodiments, the isolated nucleic acid comprises two flanking long terminal repeats (LTRs). The isolated nucleic acids of the disclosure may be recombinant adeno-associated virus (AAV) vectors (rAAV vectors).

In some embodiments, the isolated nucleic acid described herein can be packaged for delivery to a subject via a non-viral platform. In some embodiments, the isolated nucleic acid described herein can be delivered to a subject via closed-ended linear duplex DNA (ceDNA). Delivery of a transgene via ceDNA has been described previously, see e.g., WO2017152149, the entire contents of which are incorporated herein by reference. In some embodiments, the nucleic acids having asymmetric terminal sequences (e.g., asymmetric interrupted self-complementary sequences) form closed-ended linear duplex DNA structures (e.g., ceDNA) that, in some embodiments, exhibit reduced immunogenicity compared to currently available gene delivery vectors. In some embodiments, ceDNA behaves as linear duplex DNA under native conditions and transforms into single-stranded circular DNA under denaturing conditions. Without wishing to be bound by any particular theory, ceDNA are useful, in some embodiments, for the delivery of a transgene to a subject.

### Recombinant Adeno-Associated Viruses (rAAVs)

In some aspects, the disclosure provides isolated adeno-associated viruses (AAVs). As used herein with respect to AAVs, the term “isolated” refers to an AAV that has been artificially produced or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities (e.g., tissue tropism). Thus, an rAAV having a capsid appropriate for the tissue being targeted can be selected.

In some embodiments, the rAAV of the present disclosure comprises a capsid protein containing the isolated nucleic acid described herein.

Methods for obtaining recombinant AAVs having a desired capsid protein are well known in the art. (See, for example, US 2003/0138772, the contents of which are incorporated herein by reference in their entirety). Typically the methods involve culturing a host cell which contains a nucleic acid sequence encoding an AAV capsid protein; a functional rep gene; a recombinant AAV vector composed of AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein has a tropism for ocular tissues or muscle tissue. In some embodiments, an AAV capsid protein targets ocular cell types (e.g., photoreceptor cells, retinal cells, etc.).

In some embodiments, an AAV capsid protein is of an AAV serotype selected from the group consisting of AAV1, AAV2, AAV3, AAV4, AAV5, AAV6, AAV7, AAV8, AAV9, AAV9.hr, AAVrh8, AAVrh10, AAVrh39, AAVrh43, AAV.PHP, and variants of any of the foregoing. In some embodiments, an AAV capsid protein is of a serotype derived from a non-human primate, for example AAVrh8 serotype. In some embodiments, the capsid protein is of AAV serotype 6 (e.g., AAV6 capsid protein), AAV serotype 8 (e.g., AAV8 capsid protein), AAV serotype 2 (e.g., AAV2 capsid protein), AAV serotype 5 (e.g., AAV5 capsid protein), AAV serotype 9 (e.g., AAV9 capsid protein), or AAVv66 serotype (e.g., AAVv66 capsid protein). In some embodiments, the AAV capsid protein with desired tissue tropism can be selected from AAV capsid proteins isolated from mammals (e.g., tissue from a subject). (See, for example, WO2010138263A2 and WO2018071831, the entire contents of which are incorporated herein by reference).

In some embodiments, the rAAV described herein is a single stranded AAV (ssAAV). An ssAAV, as used herein, refers to an rAAV with the coding sequence and complementary sequence of the transgene expression cassette on separate strands and are packaged in separate viral capsids.

The components to be cultured in the host cell to package an rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art.

In some embodiments, the disclosure relates to a host cell containing an isolated nucleic acid that comprises an expression cassette comprising a promoter operably linked to a transgene, wherein the transgene encodes a FKBP-rapamycin binding (FRB) domain, a epigenetic modulator, a rapamycin-binding protein (FKBP), and a DNA binding domain that specifically binds to a portion of an endogenous gene promoter sequence. In some embodiments, the host cell comprises the vector (e.g., AAV vector) or the ceDNA described herein. In some embodiments, the host further comprises a rapamycin or a rapalog. In some embodiments, the rapamycin or rapalog is present in the host cell at a concentration of between 5 and 3000 nM, or any intermediate number in between. In some embodiments, the rapamycin or rapalog is present in the host cell at a concentration of between 10 and 2000 nM, or any intermediate number in between. In some embodiments, the rapamycin or rapalog is present in the host cell at a concentration of 5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1000 nM, 1500 nM, 2000 nM, 2500 nM, or 3000 nM.

A “host cell” refers to any cell that harbors, or is capable of harboring, a substance of interest. Often a host cell is a mammalian cell. In some embodiments, a host cell is a photoreceptor cell, retinal pigment epithelial cell, keratinocyte, corneal cell, and/or a tumor cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell is a mammalian cell, a yeast cell, a bacterial cell, an insect cell, a plant cell, or a fungal cell. In some embodiments, the host cell is a neuron, a photoreceptor cell, a pigmented retinal epithelial cell, or a glial cell.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. In some embodiments, a vector is a viral vector, such as an rAAV vector, a lentiviral vector, an adenoviral vector, a retroviral vector, an anellovirus vector (e.g., Anellovirus vector as described in US20200188456A1), etc. Thus, the term includes cloning and expression vehicles, as well as viral vectors. In some embodiments, useful vectors are contemplated to be those vectors in which the nucleic acid segment to be transcribed is positioned under the transcriptional control of a promoter.

### AAV-Mediated Delivery of Transgenes for Modulation of an Endogenous Gene Expression

Aspects of the instant disclosure relate to compositions comprising a recombinant AAV comprising a capsid protein and a nucleic acid encoding an expression cassette comprising a promoter operably linked to a transgene, wherein the a transgene encodes a FKBP-rapamycin binding (FRB) domain, an epigenetic modulator, a rapamycin-binding protein (FKBP), and a DNA binding domain that specifically binds to a portion of the promoter of an endogenous gene. In some embodiments, the nucleic acid further comprises AAV ITRs.

The isolated nucleic acids, plasmids, rAAVs, and compositions comprising the isolated nucleic acid described herein, the plasmids described herein, or the rAAV described herein of the disclosure may be delivered to a subject in compositions according to any appropriate methods known in the art. For example, an rAAV, preferably suspended in a physiologically compatible carrier (e.g., in a composition), may be administered to a subject, i.e. host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human. In some embodiments, the subject is a human.

Delivery of the rAAVs to a mammalian subject may be by, for example, intraocular injection, subretinal injection, topical administration, intramuscular injection or by administration into the bloodstream of the mammalian subject. Administration into the bloodstream may be by injection into a vein, an artery, or any other vascular conduit. Non-limiting exemplary methods of intramuscular administration of the rAAV include Intramuscular (IM) Injection and Intravascular Limb Infusion. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. In some embodiments, an rAAV or a composition (e.g., composition containing the isolated nucleic acid or the rAAV) as described in the disclosure is administered by intravitreal injection. In some embodiments, an rAAV or a composition (e.g., composition containing the isolated nucleic acid or the rAAV) as described in the disclosure is administered by intraocular injection. In some embodiments, an rAAV or a composition (e.g., composition containing the isolated nucleic acid or the rAAV) as described in the disclosure is administered by subretinal injection. In some embodiments, an rAAV or a composition (e.g., composition containing the isolated nucleic acid or the rAAV) as described in the disclosure is administered by intravenous injection. In some embodiments, an rAAV or a composition (e.g., composition containing the isolated nucleic acid or the rAAV) as described in the disclosure is administered by intramuscular injection. In some embodiments, an rAAV or a composition (e.g., composition containing the isolated nucleic acid or the rAAV) as described in the disclosure is administered by intratumoral injection.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some embodiments, a composition further comprises a pharmaceutically acceptable carrier. Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, parachlorophenol, and poloxamers (non-ionic surfactants) such as Pluronic® F-68. Suitable chemical stabilizers include gelatin and albumin.

The rAAVs or the compositions (e.g., composition containing the isolated nucleic acid or the rAAV described herein) are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intravitreal delivery to the eye), intraocular injection, subretinal injection, oral, inhalation (including intranasal and intratracheal delivery), intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

An effective amount of rAAVs or composition (e.g., composition containing the isolated nucleic acid or the rAAV described herein) is an amount sufficient to target infect an animal, target a desired tissue (e.g., muscle tissue, ocular tissue, etc.). In some embodiments, an effective amount of an rAAV is administered to the subject during a pre-symptomatic stage of degenerative disease. In some embodiments, a subject is administered an rAAV or composition after exhibiting one or more signs or symptoms of degenerative disease. In some embodiments, the effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 106 to 1016 genome copies (e.g., from 1×10⁶ to 1×10¹⁶, inclusive). In some embodiments, an effective amount of an rAAV ranges between 1×10⁹ and 1×10¹⁴ genome copies of the rAAV. In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of about 10¹² to 10¹⁴ rAAV genome copies is appropriate. In some embodiments, a dosage of about 10¹³ to 10¹⁴ rAAV genome copies is appropriate. In some embodiments, a dosage of about 1×10¹², about 1.1×10¹², about 1.2×10¹², about 1.3×10¹², about 1.4×10¹², about 1.5×10¹², about 1.6×10¹², about 1.7×10¹², about 1.8×10¹², about 1.9×10¹², about 1×10¹³, about 1.1×10¹³, about 1.2×10¹³, about 1.3×10¹³, about 1.4×10¹³, about 1.5×10¹³, about 1.6×10¹³, about 1.7×10¹³, about 1.8×10¹³, about 1.9×10¹³, or about 2.0×10¹⁴ vector genome (vg) copies per kilogram (kg) of body weight is appropriate. In some embodiments, a dosage of between about 4×10¹² to 2×10¹³ rAAV genome copies is appropriate. In some embodiments a dosage of about 1.5×10¹³ vg/kg by intravenous administration is appropriate. In certain embodiments, 10¹²-10¹³ rAAV genome copies is effective to target tissues. In certain embodiments, 10¹³-10¹⁴ rAAV genome copies is effective to target tissues effective to target tissues (e.g., the eye).

In some embodiments, the rAAV described herein is administered to the subject once a day, once a week, once every two weeks, once a month, once every 2 months, once every 3 months, once every 6 months, once a year, or once in a lifetime of the subject.

An effective amount of rAAVs or composition (e.g., composition containing the isolated nucleic acid or the rAAV described herein) may also depend on the mode of administration. For example, targeting an ocular (e.g., corneal) tissue by intrastromal administration or subcutaneous injection may require different (e.g., higher or lower) doses, in some cases, than targeting an ocular (e.g., corneal) tissue by another method (e.g., systemic administration, topical administration). In some embodiments, intrastromal injection (IS) of rAAV having certain serotypes (e.g., AAV2, AAV5, AAV6, AAV6.2, AAV7, AAV8, AAV9, AAVrh.8, AAVrh.10, AAVrh.39, and AAVrh.43) mediates efficient transduction of ocular (e.g., corneal, retinal, etc.) cells. Thus, in some embodiments, the injection is intrastromal injection (IS). In some embodiments, the injection is topical administration (e.g., topical administration to an eye). In some cases, multiple doses of a rAAV are administered.

In some embodiments, rAAV compositions are formulated to reduce aggregation of AAV particles in the composition, particularly where high rAAV concentrations are present (e.g., ˜10¹³ GC/mL or more). Methods for reducing aggregation of rAAVs are well-known in the art and, include, for example, addition of surfactants, pH adjustment, salt concentration adjustment, etc. (See, e.g., Wright FR, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf-life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either intravitreally, intraocularly, subretinally, subcutaneously, intraopancreatically, intranasally, parenterally, intravenously, intramuscularly, intrathecally, orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that it is easily syringed. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the transgene (e.g., FRAP-zinc finger domain fusion protein and FRB-epigenetic modulator fusion protein) described herein is delivered to the subject by ceDNA. Any compositions containing ceDNA described herein are also within the scope of the present disclosure. In some embodiments, the ceDNA and the compositions thereof can be administered to the subject using any suitable method described herein. In some embodiments, delivery of an effective amount of the ceDNA described herein by injection is in an amount such that it is sufficient to express an effective amount of an ceDNA encoding the transgene (e.g., FRAP-zinc finger domain fusion protein and FRB-epigenetic modulator fusion protein). In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, Solu-Medrol, Lansoprazole, trimethoprim/sulfamethoxazole, methotrexate, and any combination thereof. In some embodiments, the immunosuppression regimen comprises administering sirolimus, prednisolone, lansoprazole, trimethoprim/sulfamethoxazole, or any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, the methods described in this disclosure further comprise co-administration or prior administration of an agent to a subject administered an rAAV or pharmaceutical composition comprising an rAAV of the disclosure. In some embodiments, the agent is selected from a group consisting of Miglustat, Keppra, Prevacid, Clonazepam, and any combination thereof.

In some embodiments, immunosuppression of a subject maintained during and/or after administration of a rAAV or pharmaceutical composition. In some embodiments, a subject is immunosuppressed (e.g., administered one or more immunosuppressants) for between 1 day and 1 year after administration of the rAAV or pharmaceutical composition.

### Methods

Aspects of the disclosure relate to methods for treating a disease in a subject in need thereof, the method comprising: (i) administering to the subject a therapeutically effective amount of an isolated nucleic acid, vector, recombinant lentivirus, rAAV, ceDNA, host cell or pharmaceutical composition as described herein; and (ii) administering to the subject an effective amount of rapamycin or a rapalog.

In some embodiments, the isolated nucleic acid, rAAV, or composition described herein can be used to alter the chromatin structure of an endogenous gene by epigenetic modulation to treat a disease. Non-limiting examples of epigenetic-associated diseases and their associated endogenous gene promoter include: Fragile X syndrome associated with FMR-1 promoter, Rett syndrome associated with MeCP2 promoter, Schizophrenia associated with RELN promoter, Schizophrenia associated with SOX10 promoter, Schizophrenia associated with GAD1 promoter, Schizophrenia associated with WDR18 promoter, bipolar II associated with PPIEL promoter, Behcet's disease associated with RPL39 promoter, or dementia associated with CRY1 promoter. Other Diseases related to epigenetic changes of a responsible endogenous gene promoter have been previously described, for example, by Simmons et al., Epigenetic Influences and Disease, Epigenetic influence and disease. Nature Education 1(1):6; Moosavi et al., Role of Epigenetics in Biology and Human Diseases, Iran Biomed J. 2016 November; 20(5): 246-258; Zoghbi et al., Epigenetics and Human Disease, Cold Spring Harb Perspect Biol 2016; 8:a019497. In some embodiments, an endogenous gene is genetically mutated (e.g., loss of function mutation), and its endogenous analog can be activated by the isolated nucleic acid, the AAV or the composition described herein as surrogate of the mutated gene. For example, Congenital Muscular Dystrophy type 1A (MDC1A) is a genetic disease associated with a mutation in LAMA2 gene, and epigenetic activation of expression of its analog gene LAMA1, which is normally expressed only during embryonic state, can be used to treat MDC1A (see, e.g., Kemaladewi et al., A mutation-independent approach for muscular dystrophy via upregulation of a modifier gene, Nature volume 572, pages 125-130(2019)).

Another aspects of the disclosure relates to A method for treating MDC1A in a subject in need thereof, the method comprising: (i) administering to the subject a therapeutically effective amount of the isolated nucleic acid, the vector, the recombinant lentivirus, the rAAV, the ceDNA, the host cell or the pharmaceutical composition as described herein; and (ii) administering to the subject an effective amount of rapamycin or rapalog. In some embodiments, the rAAV encodes a FKBP-zinc finger domain fusion protein, and a FRB-histone demethylase fusion protein. In the presence of rapamycin or rapalog, the FKBP-zinc finger domain fusion protein and a FRB-histone demethylase fusion protein dimerizes, the zinc finger domain binds to the promoter of LAMA1 gene, and the histone demethylase can open the chromatin structure of the LAMA1 gene thus activating its transcription and expression.

In some embodiments, the subject is a human. In some embodiments, the subject is a non-human mammal. Non-limiting examples of non-human mammals are mouse, rat, cat, dog, sheep, rabbit, horse, cow, goat, pig, guinea pig, hamster, chicken, turkey, or a non-human primate.

Alleviating a disease (e.g., MDC1A) includes delaying the development or progression of the disease, or reducing disease severity. Alleviating the disease does not necessarily require curative results. As used therein, “delaying” the development of a disease (e.g., MDC1A) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In some embodiments, the level of rapamycin or rapalog does not induce immunosuppression in the subject.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

In some embodiments, the isolated nucleic acid, the vector, the recombinant lentivirus, the rAAV, the ceDNA, the host cell or the pharmaceutical composition as described herein can be administered concurrently with the rapamycin or rapalog. In some embodiments, the isolated nucleic acid, the vector, the recombinant lentivirus, the rAAV, the ceDNA, the host cell or the pharmaceutical composition as described herein can be administered sequentially with the rapamycin or rapalog. In some embodiments, the isolated nucleic acid, the vector, the recombinant lentivirus, the rAAV, the ceDNA, the host cell or the pharmaceutical composition as described herein can be administered at different frequencies with the administration of rapamycin or rapalog.

Another aspects of the disclosure also provides method for modulating an endogenous gene expression in a subject, the method comprising: (i) administering to the subject a therapeutically effective amount of the isolated nucleic acid, the vector, the recombinant lentivirus, the rAAV, the ceDNA, the host cell or the pharmaceutical composition as described herein; (ii) measuring expression of transgene in the subject; (iii) administering more or less rapalog based on expression level measured in (ii). The expression of the endogenous gene may be modulated by the concentration of rapamycin or rapalog. In some embodiments, if the level of the endogenous gene is lower than a control expression level in the subject. A control expression level of the endogenous gene is determined by the pharmacodynamics and pharmacokinetics of the transgene product, and is known in the art. In some embodiments, if the level of the endogenous gene produced is higher than the control expression level, the amount of rapamycin or rapalog can be decreased. In some embodiments, if the level of the endogenous gene produced is lower than the control expression level, the amount of rapamycin or rapalog can be increased. The level of the endogenous in the subject can be measured using routine methods in the art, such as RNA solution hybridization, nuclease protection, Northern hybridization, reverse transcription, gene expression monitoring with a microarray, antibody binding, enzyme linked immunosorbent assay (ELISA), nucleic acid sequencing, Western blotting, radioimmunoassay (RIA), other immunoassays, fluorescence activated cell analysis (FACS), or any other technique or combination of techniques that can detect the level of the endogenous gene (e.g., in a subject or a sample obtained from a subject). In some embodiments, a higher level of the endogenous gene is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a desired level of the endogenous gene. In some embodiments, a lower level of the endogenous gene is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more lower than a desired level of the endogenous gene.

In some embodiments, the amount of rapamycin or rapalog administered to the subject can be adjusted/modulated according to the level of transgene in the subject. In some embodiments, the level of rapamycin or rapalog does not induce immunosuppression in the subject. In some embodiments, when the expression level of the endogenous gene is higher relative to the control level, the concentration of rapalog administered to the subject can be the same or decreased. In some embodiments, the concentration of rapalog can be decreased at least 10%, 15%, 20%, 25%, 30%, 35%, 40%, 45%, 50% or more. In some embodiments, when the expression level of the endogenous gene is lower relative to the control level, the concentration of rapalog administered to the subject can be increased. In some embodiments, the concentration of rapalog can be increase at least 10%, 15%, 20%, 25%, 30%, 35%, 40%, 45%, 50%, 60%, 70%, 80%, 90%, 100%, 2 fold, 3 fold, 4 fold, 5 fold, 6 fold, 7 fold, 8 fold, 9 fold, 10 fold or more.

### EXAMPLES

### Example 1: Rapalog-Mediated Inducible AAV Construct for the Epigenetic Modulation of an Endogenous Gene

This example describes an inducible AAV construct dependent on rapamycin/rapalog for the regulation of a promoter of an endogenous gene to direct the expression of the endogenous gene. The single recombinant AAV vector approach to regulated gene silencing or gene expression with non-immunogenic human-based promoter elements should have broad impact and utility in the field of gene therapy.

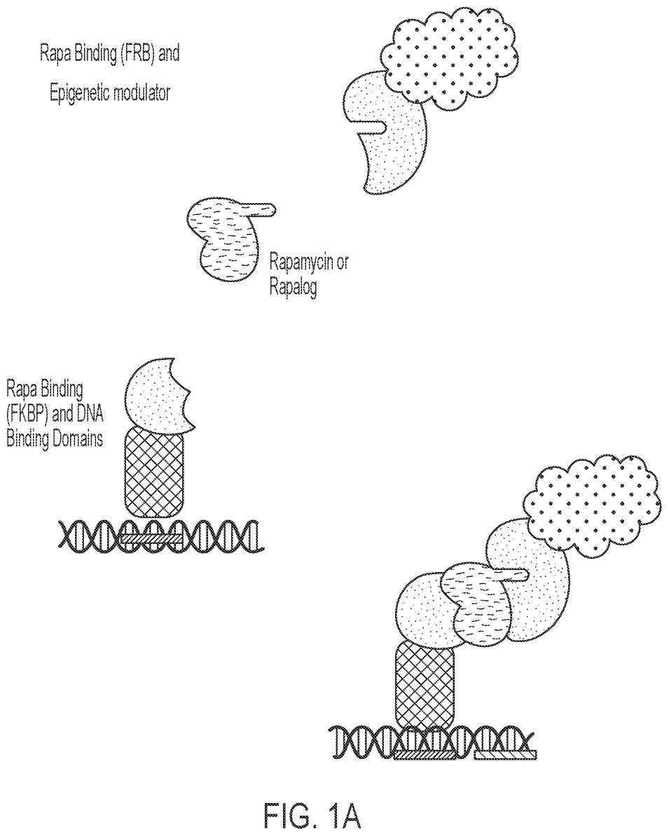

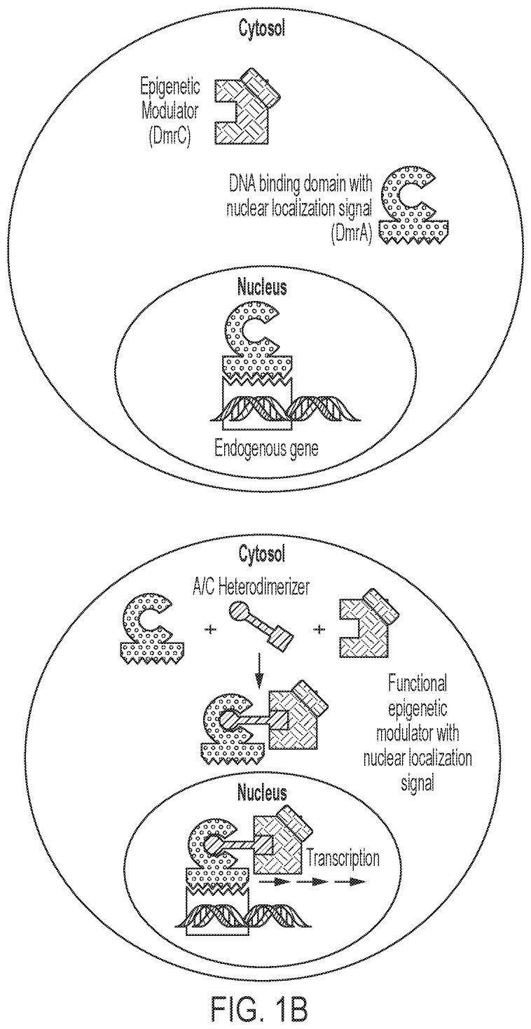

This approach relies on the heterodimerization of two different binding motifs that in the presence of a rapalog ligand complete the assembly of a complex at the endogenous gene promoter (see FIG. 1A-1B). The components are based on the human protein FKBP12 (FKBP, for FK506 binding protein) and its small molecule ligands. FKBP is an abundant cytoplasmic protein that serves as the initial intracellular target for FK506 and rapamycin. Rapamycin functions by binding with high affinity to FKBP, and then to the large PI3K homolog FRAP (RAFT, mTOR), thereby acting as a heterodimerizer to join the two proteins together. To use rapamycin to induce heterodimers epigenetic modulation complex, the zinc finger (DNA binding domain) is fused to FKBP domains, and the epigenetic modulator is fused to a 93 amino acid portion of FRAP, termed FRB, that is sufficient for binding the FKBP-rapamycin complex (see FIG. 1B). The system functions with rapamycin or the newly engineered rapalogs. The rapalogs have been chemically modified so that they no longer can bind to wild-type endogenous FRAP, greatly reducing immunosuppressive activity.

### Equivalents

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An isolated nucleic acid comprising an expression cassette comprising a promoter operably linked to a transgene, wherein the transgene encodes a FKBP-rapamycin binding protein (FRB), an epigenetic modulator, a rapamycin-binding protein (FKBP), and a DNA binding protein that specifically binds to an endogenous gene promoter.

2. The isolated nucleic acid of claim 1, wherein the promoter is a constitutive promoter, an inducible promoter, or a tissue specific promoter.

3. The isolated nucleic acid of claim 1 or 2, wherein the promoter is a minimal promoter.

4. The isolated nucleic acid of claim 2, wherein the promoter is a chicken beta-actin promoter, a Human cytomegalovirus (CMV) promoter, or a chimeric CMV-chicken β-actin (CBA) promoter.

5. The isolated nucleic acid of any one of claims 1-4, wherein the FKBP is FKBP12.

6. The isolated nucleic acid of any one of claims 1-5, wherein the DNA binding domain is a zinc finger domain, or dCas protein.

7. The isolated nucleic acid of claim 6, wherein the DNA binding domain is a zinc finger domain.

8. The isolated nucleic acid of any one of claims 1-7, wherein the FKBP is directly fused to the DNA binding domain.

9. The isolated nucleic acid of any one of claims 1-7, wherein the FKBP is fused to the DNA binding domain via a linker.

10. The isolated nucleic acid of claim 9, wherein the linker is a polypeptide linker.

11. The isolated nucleic acid of any one of claims 8-10, wherein the FKBP-DNA binding domain fusion protein is a FKBP12-zinc finger domain fusion protein.

12. The isolated nucleic acid of claim 11, wherein the FKBP12-zinc finger domain fusion protein comprises an amino acid sequence at least 80% identical to amino acid sequence of SEQ ID NO: 1.

13. The isolated nucleic acid of any one of claims 1 to 12, wherein the FRB domain is a FKBP12-rapamycin-associated protein (FRAP) domain.

14. The isolated nucleic acid of any one of claims 1 to 13, wherein the epigenetic modulator is a histone demethylases, a histone methyltransferases, a histone deacetylases, a histone acetyltransferases, a bromodomain-containing proteins, a kinase, or an actin-dependent regulators of chromatin.

15. The isolated nucleic acid of any one of claims 1 to 14, wherein FRB domain is directly fused to the epigenetic modulator.

16. The isolated nucleic acid of claim 14 or 15, wherein the epigenetic modulator is a histone demethylase.

17. The isolated nucleic acid of any one of claims 1-16, wherein the FRB is fused to the epigenetic modulator via a linker.

18. The isolated nucleic acid of claim 17, wherein the linker is a polypeptide linker.

19. The isolated nucleic acid of any one of claims 15-18, wherein the isolated nucleic acid further comprises a IRES or a 2A peptide coding sequence, wherein the IRES or 2A is located between the FKBP12-zinc finger protein fusion protein and the FRB-epigenetic modulator fusion protein.

20. The isolated nucleic acid of any one of claims 1-19, wherein the transgene each further comprises a 3′ untranslated region (3′UTR).

21. The isolated nucleic acid of any one of claims 1-20, wherein the transgene further comprises one or more miRNA binding sites.

22. The isolated nucleic acid of claim 21, wherein the one or more miRNA binding sites are positioned in a 3′UTR of the transgene.

23. The isolated nucleic acid of claim 21 or 22, wherein the at least one miRNA binding site is an immune cell-associated miRNA binding site or a liver-cell associated miRNA binding site.

24. The isolated nucleic acid of claim 23, wherein the immune cell-associated miRNA is selected from: miR-106, miR-125a, miR-125b, miR-126a, miR-142, miR-146a, miR-15, miR-150, miR-155, miR-16, miR-17, miR-18, miR-181a, miR-19a, miR-19b, miR-20, miR-21a, miR-223, miR-24-3p, miR-29a, miR-29b, miR-29c, miR-302a-3p, miR-30b, miR-33-5p, miR-34a, miR-424, miR-652-3p, miR-652-5p, miR-9-3p, miR-9-5p, miR-92a, and miR-99b-5p.

25. The isolated nucleic acid of claim 23, wherein the liver cell-associated miRNA is miR-122.

26. The isolated nucleic acid of any one of claims 1-25, wherein the endogenous gene is LAMA1.

27. The isolated nucleic acid of any one of claims 1 to 26, further comprising two flanking long terminal repeats (LTRs).

28. The isolated nucleic acid of any one of claims 1 to 26, further comprising two flanking adeno-associated virus inverted terminal repeats (ITRs).

29. The isolated nucleic acid of claim 28, wherein the ITRs are adeno-associated virus ITRs of a serotype selected from the group consisting of AAV1 ITR, AAV2 ITR, AAV3 ITR, AAV4 ITR, AAV5 ITR, and AAV6 ITR.

30. The isolated nucleic acid of any one of claims 1 to 26, wherein the isolated nucleic acid is a closed-ended linear duplex DNA (ceDNA).

31. A vector comprising the isolated nucleic acid of any one of claims 1 to 30.

32. The vector of claim 31, wherein the vector is a plasmid, a lentiviral vector, a retroviral vector, an anellovirus vector, or an adeno-associated virus (AAV) vector.

33. A recombinant adeno-associated virus (rAAV) vector comprising, in 5′ to 3′ order:
(a) a 5′ AAV ITR;
(b) a promoter;
(c) a transgene comprising, in 5′ to 3′ order:
(A) a nucleotide sequence encoding a FRB-epigenetic modulator fusion protein;
(B) a nucleotide sequence encoding an internal ribosome entry site (IRES); and
(C) a nucleotide sequence encoding a FKBP-zinc finger domain fusion protein; and
(d) a 3′ AAV ITR.

34. A recombinant lentivirus comprising a lentiviral capsid containing the isolated nucleic acid of any one of claims 1 to 30.

35. A recombinant adeno-associated virus (rAAV) comprising:
(i) an isolated nucleic acid of any one of claim 1-26, 28, or 29; and
(ii) at least one AAV capsid protein.

36. The rAAV of claim 35, wherein the capsid protein is of a serotype selected from AAV1, AAV2, AAV3, AAV4, AAV5, AAV6, AAV7, AAV8, AAV9 and a variant of any of the foregoing.

37. The rAAV of claims 35 of 36, wherein the rAAV is a single-stranded AAV (ssAAV).

38. A recombinant adeno-associated virus (rAAV), comprising:
(i) an AAV capsid protein; and
(ii) an isolated nucleic acid comprising, in 5′ to 3′ order:
(a) a 5′ AAV ITR;
(b) a promoter;
(c) a transgene comprising, in 5′ to 3′ order:
(A) a nucleotide sequence encoding a FRB-epigenetic modulator fusion protein;
(B) a nucleotide sequence encoding an internal ribosome entry site (IRES); and
(C) a nucleotide sequence encoding a FKBP-zinc finger domain fusion protein; and
(d) a 3′ AAV ITR.

39. A host cell comprising:
(i) isolated nucleic acid of any one of claims 1-30, the vector of any one of claims 31-33, the recombinant lentivirus of claim 34, or the rAAV of claims 35-38; and
(ii) rapamycin or a rapalog.

40. The host cell of claim 39, wherein the host cell is a mammalian cell, yeast cell, bacterial cell, or insect cell.

41. The host cell of claim 39 or 40, wherein the rapalog is Sirolimus, Temsirolimus, or Everolimus.

42. The host cell of any one of claims 39-41, wherein the concentration of the rapalog is 10 nM to 2000 nM.

43. A pharmaceutical composition comprising the isolated nucleic acid of any one of claims 1-30, the vector of any one of claims 31-33, the recombinant lentivirus of claim 34, the rAAV of claims 35-38, or the host cell of any one of claims 39-42.

44. The pharmaceutical composition of claim 43, further comprises a pharmaceutically acceptable carrier.

45. The pharmaceutical composition of claim 43 or 44, wherein the pharmaceutical composition is formulated for intravenous injection, intraperitoneal injection, intracranial injection, intratumoral injection, intramuscular injection, or intravitreal injection.

46. A method for treating a disease in a subject in need thereof, the method comprising:
(i) administering to the subject a therapeutically effective amount of the isolated nucleic acid of any one of claims 1-30, the vector of any one of claims 31-33, the recombinant lentivirus of claim 34, the rAAV of claims 35-38, the host cell of any one of claims 39-42, or the pharmaceutical composition of any one of claims 43-45; and
(ii) administering to the subject an effective amount of rapamycin or a rapalog.

47. A method for treating Congenital Muscular Dystrophy type 1A (MDC1A) in a subject in need thereof, the method comprising:
(i) administering to the subject a therapeutically effective amount of the isolated nucleic acid of any one of claims 1-30, the vector of any one of claims 31-33, the recombinant lentivirus of claim 34, the rAAV of claims 35-38, the host cell of any one of claims 39-42, or the pharmaceutical composition of any one of claims 43-45; and
(ii) administering to the subject an effective amount of rapamycin or a rapalog.

48. The method of claim 46 or 47, wherein the zinc finger domain is capable of binding to endogenous LAMA1 promoter.

49. The method of any one of claims 45-48, wherein the subject is a non-human mammal.

50. The method of claim 49, wherein the non-human mammal is mouse, rat, cat, dog, sheep, rabbit, horse, cow, goat, pig, guinea pig, hamster, chicken, turkey, or a non-human primate.

51. The method of any one of claims 45-48, wherein the subject is a human.

52. The method of any one of claims 45-51, wherein the administration of (i) or (ii) is intravenous injection, intraperitoneal injection, intracranial injection, intratumoral injection, intramuscular injection, or intravitreal injection.

53. The method of any one of claims 45-52, wherein administering (i) and (ii) is concurrent.

54. The method of any one of claims 45-52, wherein administering (i) and (ii) is sequential.

55. The method of any one of claims 45-52, wherein administering (i) and (ii) is at different frequencies.

56. The method of claim 55, wherein (i) is administered once and (ii) is administered repeatedly.

57. The method of claim 56, wherein (ii) is administered every week, every two weeks, or every month.

58. The method of any one of claims 45-57, wherein the dose of rapamycin or rapalog does not induce immunosuppression in the subject.

59. A method for modulating endogenous expression in a subject, the method comprising:
(i) administering the isolated nucleic acid of any one of claims 1-30, the vector of any one of claims 31-33, the recombinant lentivirus of claim 34, the rAAV of claims 35-38, the host cell of any one of claims 39-42, or the pharmaceutical composition of any one of claims 43-45, and a rapalog to a subject;
(ii) measuring expression of transgene in the subject relative to a control expression level of the endogenous gene;
(iii) adjusting the dose of rapalog-based on expression level measured in (ii), wherein if the expression level measured in (ii) is increased relative to the control level, administering the same or less concentration of the rapalog; and wherein if the expression level measured in (ii) is the same or decreased relative to the control level, administering a higher concentration of the rapalog to the subject.


SEQUENCE LISTING (1 sequence)

SEQ ID NO: 1 - protein, 465 aa, Artificial Sequence
  Synthetic
MDYPAAKRVKLDSRERPYACPVESCDRRFSRSDELTRHIRIHTGQKPFQCRICMRNFSRSDHLTTHIRTHTGGGRRRKKRTSIETNIRVALEKSFLENQKPTSEEITMIADQLNMEKEVIRVWFCNRRQKEKRINTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY